(19) United States
(12) Patent Application Publication
(10) Pub. No.: US 2006/0212057 A1
(43) Pub. Date: Sep. 21, 2006

(54) Miniature surgical scalpel with integral protection shield

(76) Inventors: Ilija Djordjevic, East Granby, CT (US); Sushil K. Kanwar, West Hartford, CT (US)

Correspondence Address: ALIX YALE & RISTAS LLP, 750 MAIN STREET, SUITE 1400, HARTFORD, CT, 06103, US

(21) Appl. No.: 11/374,389

(22) Filed: Mar. 13, 2006

Related U.S. Application Data
(60) Provisional application No. 60/663,376, filed on Mar. 18, 2005.

(51) Int. Cl. A61B 17/32

(52) U.S. Cl. 606/167

(57) ABSTRACT

A miniature scalpel comprising a carrier having a front portion rigidly securing a projecting blade and pivotally securing the back portion of a shield that projects alongside and covers one side of the blade. The front portion of the shield can be pivoted 180 degrees away from the blade into lateral alignment with the back portion of the carrier, thereby forming a handle for the exposed blade. Detent formations can be provided on the carrier and shield, for mating as the handle is formed. The carrier shield can easily be separated from the carrier at the detent, for pivoting back to the closed position.

### RELATED APPLICATION

This application claims priority under 35 USC §119(e) from U.S. Application Ser. No. 60/663,376, filed Mar. 18, 2005.

### BACKGROUND OF THE INVENTION

The present invention relates to surgical scalpels, and particularly to miniature scalpels.

Miniature surgical scalpels for use in constrained space applications, so-called “thumb scalpels”, are already available on the market. However, in order to protect the surgeon and other handling personnel from injury, these scalpels are equipped with removable protection sheaths. These protection sheaths can be temporarily lost in the process of surgery and consequently no longer provide the intended protection against injury or contamination if the scalpel is to be reused during the same surgical procedure.

### SUMMARY OF THE INVENTION

The present invention is directed to an improved miniature scalpel with integral protection shield.

In one embodiment the miniature scalpel comprises a carrier having a forward portion rigidly securing a substantially flat, projecting blade and pivotally securing the back portion of a shield that projects alongside and covers one side of the blade. The forward portion of the shield can be pivoted 180 degrees away from the blade into lateral alignment with the back portion of the carrier, thereby forming a handle for the exposed blade. The shield is preferably biased toward the carrier, such that the forward portion of the shield is urged laterally against the blade when the scalpel is closed, to enhance the covering of the blade, and laterally against the aligned back portion of the carrier when the scalpel is open, to enhance the integrity of the formed handle.

Another embodiment is directed to a miniature scalpel which in the closed position comprises a carrier having a forward portion rigidly securing a projecting blade and pivotally securing the back portion of a shield that projects alongside the blade. Detent structure or the like are provided on the back portion of the carrier and on the forward portion of the shield, whereby the forward portion of the shield can be pivoted 180 degrees away from the blade into congruent lateral alignment with the back portion of the carrier, thereby forming a handle for the exposed blade. The carrier shield can easily be separated from the carrier at the detent, for pivoting back to the closed position.

### BRIEF DESCRIPTION OF THE DRAWING

The preferred embodiment of the invention will be described with reference to the accompanying drawings, in which:

FIGS. 1A, 1B, and 1C show the steps for the subassembly of the blade and blade carrier, with the blade on the right side of the carrier;

FIG. 2 shows the subsequent step of joining the protective shield to the subassembly of blade and carrier;

FIGS. 3A and 3B show the steps for final assembly;

FIG. 4 shows the device with the shield pivoted 180 degrees clockwise relative to the shield of the device depicted in FIG. 3B, to expose the blade for use during surgery;

FIG. 5 shows how the user opens the device to expose the blade;

FIG. 6 shows the device of FIG. 5 in the fully open position, and how the user closes the device; and

FIGS. 7A and B show the device of FIG. 5 in the closed condition, from the right and left lateral sides and FIGS. 7C and 7D are top views of the device shown in FIG. 7A, with the blade and with the blade omitted for clarity, respectively.

### DETAILED DESCRIPTION

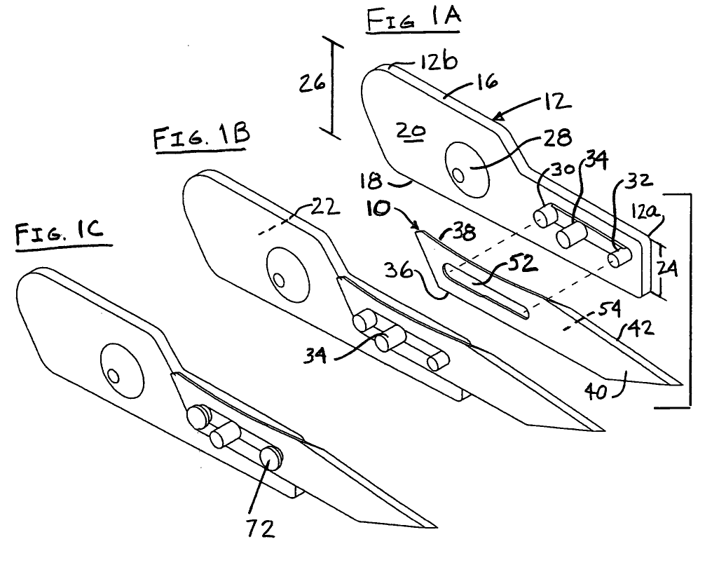

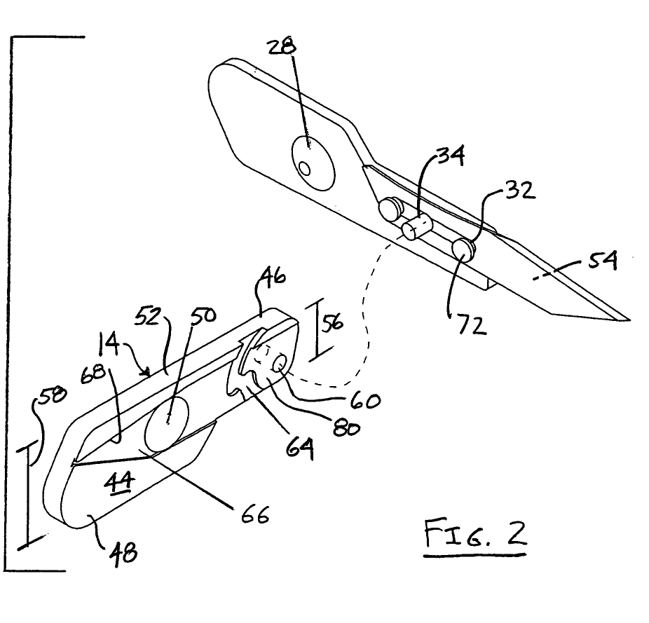

As shown in FIGS. 1-3, the inventive miniature scalpel comprises three main components: a stainless steel blade 10, a plastic blade carrier 12, and a plastic blade shield or cover 14.

The first two components and their subassembly can be best seen in FIG. 1. The carrier 12 is a substantially flat, elongated member having a forward portion 12a and a back portion 12b, top 16 and bottom 18 edges, and opposed sides 20, 22. The bottom edge 18 is substantially straight, whereas the top edge 16 bends in the approximate middle. As a result, the front portion 12a of the carrier is substantially rectangular defining a front height 24 between edges and the back portion 12b of the carrier is enlarged relative to the front portion with a back height 26 greater than the front height 24. The back portion 12b of the carrier has a first detent formation 28, and the front portion 12a has a mounting island including three longitudinally spaced, laterally projecting pins or studs defining rivets 30,32, and pivot pin 34 between them.

The blade 10 is rigidly secured to and projects from the forward portion 12a of the carrier 12. Several different shapes and sizes of suitable blades are commercially available. The blade is substantially flat, with a back portion 38 that is affixed to the front portion of the carrier and a front portion 40 with sharpened edge 42 that projects from the carrier. The opposite edge 36 is preferably substantially straight, whereas the top edge 42 can be somewhat concave where attached to the carrier, and straight where sharpened and projecting from the carrier. The blade 12 has one side 54 that closely contacts one side 20 of the carrier. The back (mounting) portion of the blade has a longitudinal through slot 52 through which the rivets 30, 32 project on either side of the pivot pin 34. The rivets 30, 32 have heads formed thereon to complete the subassembly of FIG. 1C.

The shield 14 has a back portion 46 pivotally secured to the front portion 12a of the carrier and a front portion 48 projecting from the carrier alongside the blade. The forward portion 48 of the shield has a second detent formation 50 matable with the first detent formation 28 on the carrier. The back portion of the shield is substantially rectangular defining a back height 56 between edges and the front portion of the shield is enlarged relative to the back portion of the shield with a front height 58 greater than the back height.

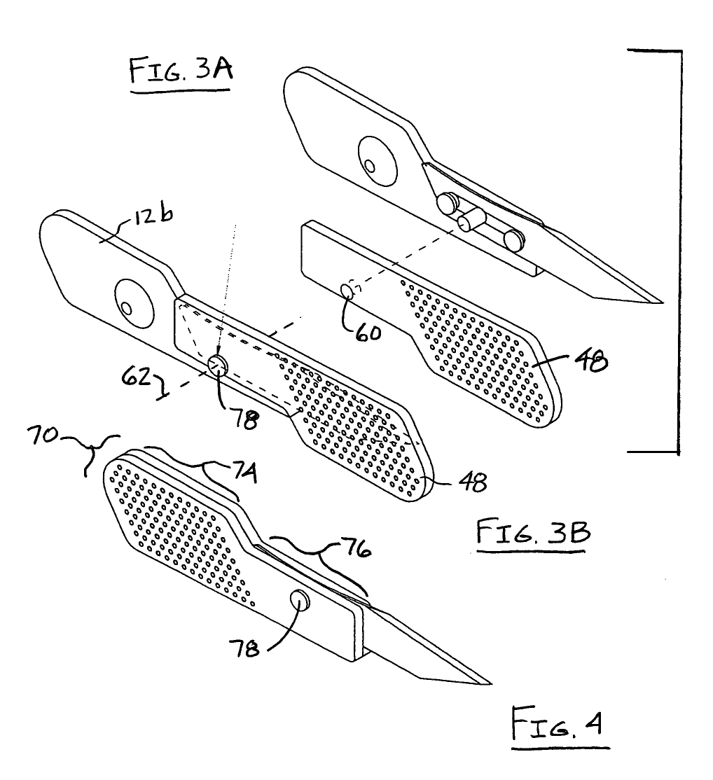

The pivot pin 34 of the carrier 12 projects rigidly from the back portion of the blade, through a hole 60 in the shield, for defining the pivot axis 62 of the shield relative to the carrier. The pivot pin 34 receives a rivet head 78 after the shield has been installed, as shown in FIG. 3B. The head 72 of the forward rivet 32 rides in an arcuate channel 64 in the shield. The channel provides a free space for the rivet during closing and opening of the device. As shown in FIG. 7B and C, the other rivet 30 is never confronted by the shield, so no structural accommodation for pivoting is required.

The shield has a recess 66 on the side 44 facing the carrier and extending from the back to the front of the shield, in which the second detent formation 50 is located and the blade is nested, such that at least the sharp edge 42 of the blade is covered by the upper rim 68 of the pocket when the device is in the closed condition shown in FIG. 3B.

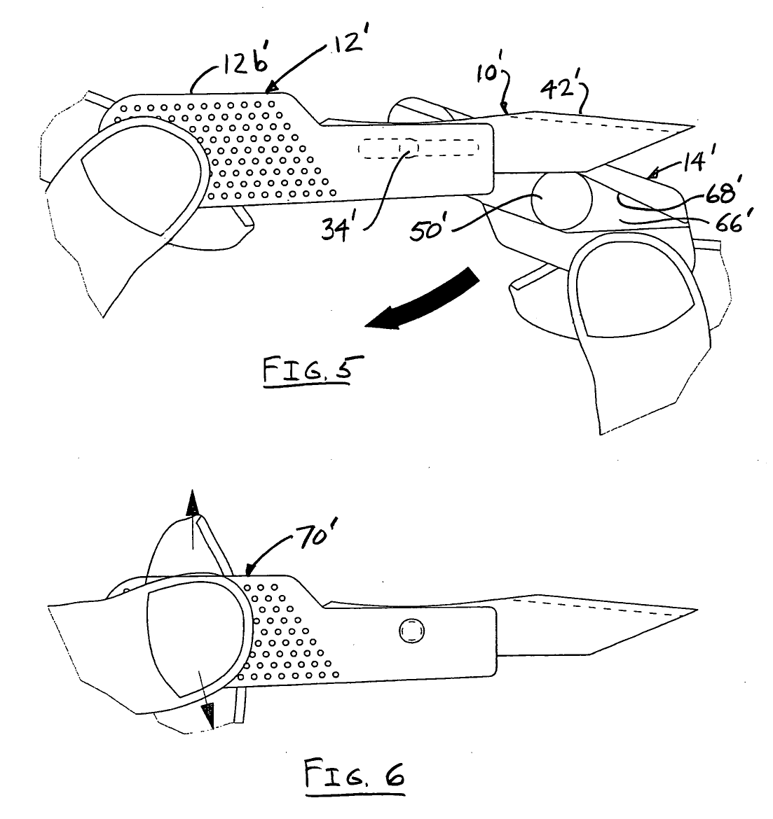

The closed scalpel can be opened to expose the blade, as shown in FIG. 4, by pivoting the forward portion 48 of the shield 180 degrees clockwise away from the blade into lateral alignment with the back portion 12b of the carrier, thereby mating the detent formations 28,50 such that the back portion of the carrier and the front portion of the shield connect via the detent structure 28,50 and together form a handle 70 for the exposed blade.

In the open condition of the scalpel, the back portion of the carrier and the front portion of the shield are substantially congruent, together forming a wider back portion 74 of the handle, and the front portion of the carrier and the back portion of the shield are substantially congruent, together forming a narrower, front portion 76 of the handle. When opened for use, the handle 70 thickens as shown in FIG. 4, and provides a very ergonomically effective combination of substantial height in the back portion 74 for steady holding in the palm and last three fingers, and reduced height in the forward portion 76 for delicate manipulation between the forefinger and thumb.

It should thus be appreciated that some of the important features depicted in FIGS. 1-3 are the preferably semi-spherical protrusion 28 forming the detent structure on the carrier, and the blade locating island with three cylindrical protrusions 30, 32, 34. The first sub-assembly is created by placing the blade over the locating island and subsequently riveting both peripheral cylindrical pins 30, 32 (ultra sound or thermal riveting) in order to firmly and permanently attach the blade to the blade carrier. The shield is then slipped over the mounting pin 34 of the blade carrier and subsequently riveted in such a way, as to retain blade protection by the shield, but at the same time to allow for shield rotation between the two positions (closed, with blade protected and open, with blade exposed). The angle between the axis 62 of the mounting hole 60 and the immediately surrounding surface 80 of the shield is slightly less then 90 degrees, creating a bias for urging the shield toward the carrier. This provides some positive lateral force in both the open and closed positions of the scalpel. This is illustrated in FIG. 7C.

After the final assembly (FIG. 3B) the miniature scalpel is sterilized and subsequently packaged in a sealed container.

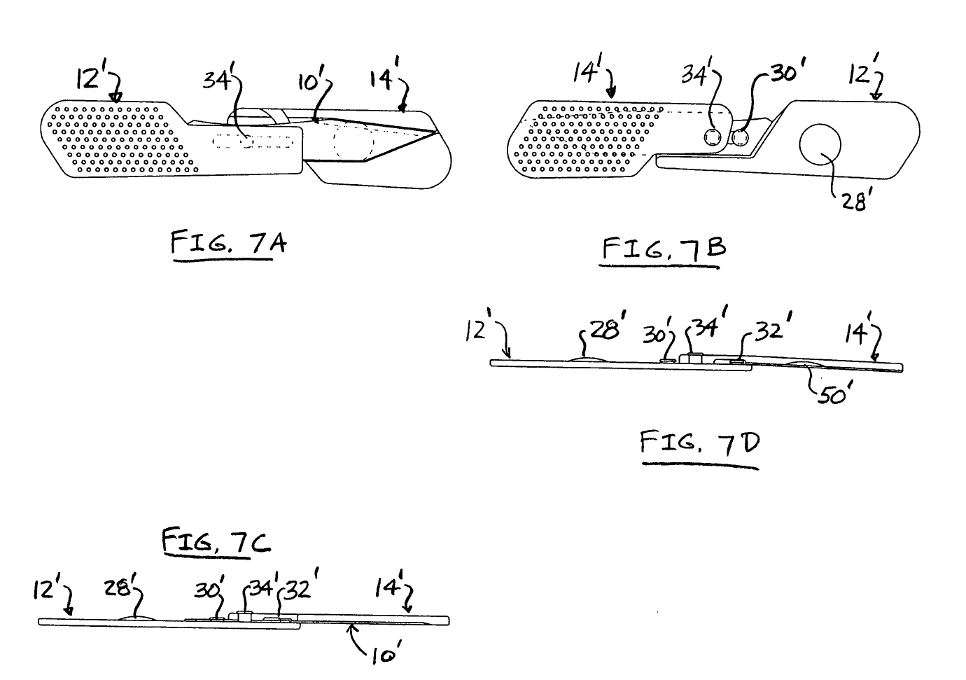

FIGS. 5 and 6 show the device in an opening and closing sequence. After the scalpel is removed from its packaging envelope the user grabs the end 12b ′ of blade carrier 12′ with one hand and the protection shield 14′ by the other hand (on the side opposite to the cutting edge 42′ of the blade) and by applying slight lateral force allows the blade 10′ to be removed from its protection pocket 66′ in the protection shield (FIG. 5). The protection shield is then rotated 180 degrees around the mounting pin 34′, which allows engagement between spherical protrusion of the blade carrier and the spherical depression 50′ in the protection shield, to provide stabile handle during the surgery. Before the miniature scalpel is temporarily set aside or permanently discarded, the blade has to be protected, by reversing the above procedure. First the halves forming handle 70′ are split by applying opposite forces between the thumb and forefinger (FIG. 6) and subsequently the protection shield is rotated by 180 degrees counter clockwise around the axis of the mounting pin, allowing for the blade to hide in its pocket 66′.

FIGS. 7A and B show the device of FIG. 5 in the closed position, from right and left lateral sides. In the closed condition, the overall profile or envelope is substantially rectangular. FIG. 7C shows a top view of the device of FIG. 7A, and FIG. 7D shows the same view with the blade omitted for clarity. These figures show that the device maintains a very slim width in the closed position.

1. A miniature scalpel comprising:
a carrier having front and back portions;
a substantially flat blade projecting from the front portion of the carrier;
a shield having a back portion pivotally secured to the carrier and a front portion projecting from the carrier along and covering one flat side of the blade;
whereby the front portion of the shield can be pivoted 180 degrees away from the blade into alignment with the back portion of the carrier, exposing the blade and forming a handle for the exposed blade.

2. The miniature scalpel of claim 1 wherein the carrier and the shield have substantially identical shapes such that when forming said handle, the back portion of the carrier and the front portion of the shield are substantially congruent, together forming a wider back portion of the handle, and the front portion of the carrier and the back portion of the shield are substantially congruent, together forming a narrower, front portion of the handle.

3. The miniature scalpel of claim 1, wherein a pivot pin projects laterally from the front portion of the carrier, through a back portion of the blade, into engagement with the shield, for defining a pivot axis of the shield relative to the carrier.

4. The miniature scalpel of claim 1, wherein the back portion of the carrier has a first detent formation and the front portion of the shield has a second detent formation matable with the first detent formation as the handle is formed.

5. The miniature scalpel of claim 1, wherein the front portion of the shield has a recess for covering said one side and the cutting edge of the blade when the shield is aligned with the blade.

6. The miniature scalpel of claim 1, wherein the shield is secured to the carrier with a bias that urges the shield toward the carrier.

7. A miniature scalpel which in the closed position comprises:
a carrier having a front portion and a back portion;
a blade rigidly secured to and projecting from the front portion of the carrier;
a shield having a back portion pivotally secured to the front portion of the carrier and having a front portion projecting from the carrier alongside the blade;
said back portion of the carrier having a first detent formation and said front portion of the shield having a second detent formation matable with the first detent formation;
whereby the closed scalpel can be opened to expose the blade, by pivoting the front portion of the shield 180 degrees away from the blade into lateral alignment with the back portion of the carrier, thereby mating the detent formations such that the back portion of the carrier and the front portion of the shield together form a handle for the exposed blade.

8. The miniature scalpel of claim 7, wherein,
the carrier is a substantially flat, elongated member having top and bottom edges and opposed sides;
the blade is affixed to one side of the carrier; and
the first detent formation is on said one side of the carrier.

9. The miniature scalpel of claim 8, wherein the shield is attached to said one side of the carrier and covers one side of the blade which faces the shield, while the front portion of the other side of the blade is exposed.

10. The miniature scalpel of claim 9, wherein,
the front portion of the carrier is substantially rectangular defining a front height between edges and the back portion of the carrier is enlarged relative to the front portion with a back height greater than the front height; and
the back portion of the shield is substantially rectangular defining a front height between edges and the front portion of the shield is enlarged relative to the back portion of the shield with a back height greater than the front height.

11. The miniature scalpel of claim 7, wherein,
the blade is substantially flat, with a back portion that is affixed to the front portion of the carrier and a front portion with sharpened edge that projects from the carrier; and
a pivot pin projects rigidly from the back portion of the blade, into engagement with the shield, for defining the pivot axis of the shield relative to the carrier.

12. The miniature scalpel of claim 11, wherein the pivot pin is a rivet that extends from the carrier through the shield and connects the shield to the carrier.

13. The miniature scalpel of claim 12, wherein,
the blade is affixed to the carrier by two longitudinally spaced rivets, each having a head facing the shield; and
the head of one rivet rides in an arcuate channel in the shield.

14. The miniature scalpel of claim 13, wherein the blade has a longitudinal through slot in which said rivets are situated on either side of the pivot pin.

15. The miniature scalpel of claim 7, wherein the shield has a recess facing the carrier and extending from the back to the front of the shield, in which said second detent formation is located and said blade is nested, such that at least the sharp edge of the blade is covered.

16. The miniature scalpel of claim 7, wherein the carrier and the shield have substantially identical shapes, such that in the open condition of the scalpel, the back portion of the carrier and the front portion of the shield are substantially congruent, together forming a wider back portion of the handle, across the edges, and the front portion of the carrier and the back portion of the shield are substantially congruent, together forming a narrower, front portion of the handle, across the edges.

17. The miniature scalpel of claim 7, wherein the carrier and the shield are connected at an angled mating surface whereby the front portion of the shield is biased against the blade.
